(19) United States
(12) Patent Application Publication
(10) Pub. No.: US 2005/0256171 A1
(43) Pub. Date: Nov. 17, 2005

(54) Pyrrolidine derivatives as tryptase inhibitors

(75) Inventors: Thomas Martin, Konstanz (DE); Wolf-Rudiger Ulrich, Konstanz (DE)

Correspondence Address: NATH & ASSOCIATES PLLC, 1030 FIFTEENTH STREET, N.W., SIXTH FLOOR, WASHINGTON, DC, 20005, US

(73) Assignee: Altana Pharma A G, Konstanz (DE)

(21) Appl. No.: 10/522,303

(22) PCT Filed: Jul. 24, 2003
(86) PCT No.: PCT/EP03/08127

§ 371 Date: Jan. 25, 2005

(30) Foreign Application Priority Data
Jul. 26, 2002 (EP) 02016683.1

(51) Int. Cl. A61K 31/4439; A61K 31/4025; A61K 31/401; C07D 43/02; C07D 49/02

(52) U.S. Cl. 514/343; 548/531; 514/423; 514/422; 548/527; 546/279.1

(57) ABSTRACT

Compounds of formula 1 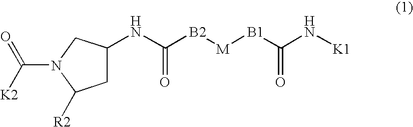 in which M, B1, B2, R2, K1 and K2 have the meanings indicated in the description are novel effective tryptase inhibitors.

### APPLICATION OF THE INVENTION

The invention relates to novel tryptase inhibitors, which are used in the pharmaceutical industry for producing pharmaceutical compositions.

### KNOWN TECHNICAL BACKGROUND

The international applications WO95/32945, WO96/09297, WO98/04537, WO99/12918, WO99/24395, WO99/24407, WO99/40073, WO99/40083, WO00/14097, WO01/10845, WO01/10848, WO01/19809, WO01/46128 and WO01/46168 describe low molecular weight bivalent compounds as tryptase inhibitors. In Clark et al., American Journal of Respiratory and Critical Care Medicine, Vol. 152, No. 6, 1995, part 1, pp. 2076-2083, the effects of two inhibitors of tryptase, APC366 [N-(1-hydroxy-2-naphthoyl)methane]-L-arginyl-L-prolinamide hydrochloride] and BABIM [bis(5-amidino-2-benzimidazolyl)methane] were examined in the allergic sheep model.

### DESCRIPTION OF THE INVENTION

It has now been found that the compounds of the formula 1 described in detail below have surprising and particularly advantageous properties.

The invention relates to compounds of the formula 1 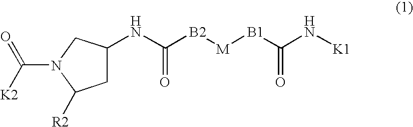
in which
M is a central building block selected from the following list 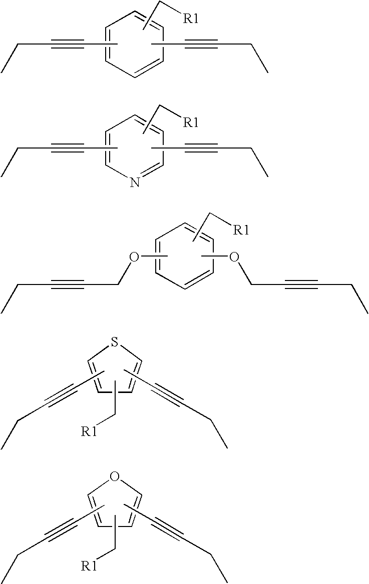
   where
R1 is hydroxycarbonyl or 1-4C-alkoxycarbonyl,
B1 and B2 are identical or different and are —O—, —NH—, —O—(CH₂)ₘ—O— or —O—(CH₂)ₘ—NH—,
m is 1, 2, 3 or 4,
K1 is —B3-Z1—B5-X1,
K2 is —B4-Z2—B6-X2,
B3 is a bond or 1-2C-alkylene,
B4 is a bond or 1-2C-alkylene,
B5 is a bond or 1-2C-alkylene,
B6 is a bond or 1-2C-alkylene,
X1 and X2 are identical or different and are amino, aminocarbonyl, amidino or guanidino,
Z1 and Z2 are identical or different and are 5,2-pyridinylene, 3,6-pyridinylene, 4,2-pyridinylene, 1,3-phenylene, 1,4-phenylene, 1,3-cyclohexylene or 1,4-cyclohexylene,
R2 is —C(O)OR3 or —C(O)N(R4)R5 where
R3 is hydrogen, 1-4C-alkyl, 3-7C-cycloalkyl, 3-7C-cycloalkylmethyl or benzyl,
R4 and R5 are, independently of one another, hydrogen, 1-4C-alkyl, 3-7C-cycloalkyl or 3-7C-cyclo-alkylmethyl, or in which R4 and R5 together and with inclusion of the nitrogen atom to which they are bonded are a 1-pyrrolidinyl, 1-piperidinyl, 1-hexahydroazepinyl, 1-piperazinyl or 4-morpholinyl radical,
   and the salts of these compounds.

1-4C-Alkyl stands for straight-chain or branched alkyl radicals having 1 to 4 carbon atoms. Examples which may be mentioned are the butyl, isobutyl, sec-butyl, tert-butyl, propyl, isopropyl, ethyl and methyl radicals.

3-7C-Cycloalkyl stands for cyclopropyl, cyclobutyl, cyclopentyl, cyclohexyl or cycloheptyl.

3-7C-Cycloalkylmethyl stands for a methyl radical which is substituted by one of the aforementioned 3-7C-cycloalkyl radicals. The 3-5C-cycloalkylmethyl radicals cyclopropylmethyl, cyclobutylmethyl, and cyclopentylmethyl may be mentioned as preferred.

1-2C-Alkylene stands for methylene [—CH₂—] or ethylene radicals, [—CH₂—CH₂—].

1-4C-Alkoxy stands for radicals which, besides the oxygen atom, comprise a straight-chain or branched alkyl radical having 1 to 4 carbon atoms. Examples which may be mentioned are the butoxy, isobutoxy, sec-butoxy, tert-butoxy, propoxy, isopropoxy and, preferably, the ethoxy and methoxy radicals.

1-4C-Alkoxycarbonyl stands for a carbonyl group to which one of the aforementioned 1-4C-alkoxy radicals is bonded. Examples which may be mentioned are the methoxycarbonyl [CH₃O—C(O)—] and the ethoxycarbonyl radical [CH₃CH₂O—C(O)—].

The definition of M comprises chemical formulae such as, for example,

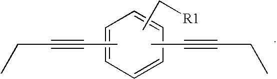

The depicted formula indicates that the radicals —CH₂—R1, —CH₂—C≡C— and —C≡C—CH₂— can be linked in any desired combination with the benzene ring.

The groups Z1 and Z2 are by definition located between the groups B3 and B5 (—B3-Z1—B5-), and B4 and B6 (—B4-Z2—B6-), respectively. Correspondingly, in the divalent groups mentioned by way of example (e.g. 4,2-pyridinylene), the first number represents the point of linkage with the group B3 or B4 and the second number represents the point of linkage with the group B5 or B6.

The groups Z1 and Z2 may assume inter alia the meaning of 1,4-cyclohexylene and 1,3-cyclohexylene. The invention includes both compounds of the formula 1 in which the groups B3, B5 or B4, B6 are linked (1e,4e)-, (1a,4a)-, (1e,4a)-, (1a,4e)-, (1e,3e)-, (1a,3a)-, (1e,3a)- and (1a,3e)- to the cyclohexylene radical. Preference is given in this connection in particular to the (1 e,4e) linkage (“e” means equatorial and “a” means axial).

Various configurations are possible in these substituted pyrrolidine building blocks of the compounds of the formula 1. These are referred to as (2S, 4S)—, (2R, 4R)—, (2S, 4R)— and (2R, 4S)—, according to the nomenclature of Cahn, Ingold and Prelog. The Invention includes compounds of the formula 1 which may comprise pyrrolidine building blocks with each of these configurations. Preferred compounds of the formula 1 are those in which the configuration on the pyrrolidine building block is (2S,4S).

Suitable salts for compounds of the formula 1 are—depending on the substitution—acid addition salts and salts with bases. Particular mention may be made of the pharmacologically acceptable salts of the inorganic and organic acids normally used in pharmaceutical technology. Suitable as such are, on the one hand, water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, with the acids being employed to prepare the salts in a ratio of amounts which is equimolar or different therefrom—depending on whether the acid is monobasic or polybasic and depending on which salt is desired.

On the other hand, salts with bases are also suitable. Examples of salts with bases which may be mentioned are alkali metal (lithium, sodium, potassium) or calcium, aluminum, magnesium, titanium, ammonium, meglumine or guanidinium salts, with the bases being employed to prepare these salts once again in a ratio of amounts which is equimolar or different therefrom.

Pharmacologically unacceptable salts, which may be for example the initial products of the process for preparing the compounds of the invention on the industrial scale, are converted into pharmacologically acceptable salts by processes known to the skilled worker.

The skilled worker is aware that the compounds of the invention, as well as their salts, may contain various amounts of solvents when they are isolated for example in crystalline form. The invention therefore also includes all solvates and, in particular, all hydrates of the compounds of the formula 1, and all solvates and, in particular, all hydrates of salts of the compounds of the formula 1.

Compounds of the formula 1 which are to be emphasized are those in which
M is the following central building block 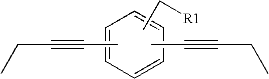
   where
R1 is hydroxycarbonyl or 1-4C-alkoxycarbonyl,
B1 and B2 are identical or different and are —O— or —O—(CH₂)ₘ—O—,
m is 2,
K1 is —B3-Z1—B5-X1,
K2 is —B4-Z2—B6×2,
B3 is a bond or 1-2C-alkylene,
B4 is a bond or 1-2C-alkylene,
B5 is a bond or 1-2C-alkylene,
B6 is a bond or 1-2C-alkylene,
X1 and X2 are identical or different and are amino or amidino,
Z1 is 3,6-pyridinylene, 4,2-pyridinylene, 1,3-phenylene or 1,4-phenylene,
Z2 is 1,3-phenylene or 1,4-phenylene,
R2 is —C(O)OR3 where
R3 is hydrogen, 1-4C-alkyl, 3-7C-cycloalkyl, 3-7C-cycloalkylmethyl or benzyl,
   and the salts of these compounds.

Compounds of the formula 1 to be particularly emphasized are those in which
M is the following central building block 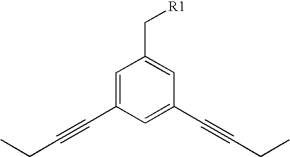
   where
R1 is methoxycarbonyl,
B1 and B2 are identical and are —O—,
K1 is —B3-Z1—B5-X1,
K2 is —B4-Z2—B6-X2,
B3 is methylene,
B4 is ethylene,
B5 is a bond or methylene,
B6 is methylene,
X1 and X2 are identical and are amino,
Z1 is 3,6-pyridinylene, 4,2-pyridinylene, 1,3-phenylene or 1,4-phenylene,
Z2 1,3-phenylene or 1,4-phenylene,
R2 is methoxycarbonyl,
   and the salts of these compounds.

Preferred compounds of the formula 1 are methyl 4-(3-{3-[3-(3-aminomethylbenzylcarbonyloxy)prop-1-ynyl]-5-methoxycarbonylmethylphenyl}prop-2-ynyloxycarbonylamino)-1-[3-(3-aminomethylphenyl)propanoyl]pyrrolidine-2-carboxylate, methyl 4-(3-{3-[3-(3-aminomethylbenzylcarbonyloxy)prop-1-ynyl]-5-methoxycarbonylmethylphenyl}prop-2-ynyloxycarbonylamino)-1-[3-(4-aminomethylphenyl)propanoyl]pyrrolidine-2-carboxylate, methyl 1-[3-(3-aminomethylphenyl)propanoyl]-4-(3-{3-[3-(6-aminopyridin-3-ylmethylcarbonyloxy)prop-1-ynyl]-5-methoxycarbonylmethylphenyl}prop-2-ynyloxycarbonylamino)pyrrolidine-2-carboxylate, methyl 1-[3-(4-aminomethylphenyl)propanoyl]4-(3-{3-[3-(6-aminopyridin-3-ylmethylcarbonyloxy)prop-1-ynyl]-5-methoxycarbonylmethylphenyl}prop-2-ynyloxycarbonylamino)pyrrolidine-2-carboxylate, methyl 1-[3-(4-aminomethylphenyl)propanoyl]-4-(3-{3-[3-(2-aminopyridin-4-ylmethylcarbonyloxy)prop-1-ynyl]-5-methoxycarbonylmethylphenyl}prop-2-ynyloxycarbonylamino)pyrrolidine-2-carboxylate, methyl 1-[3-(3-aminomethylphenyl)propanoyl]-4-(3{3-[3-(2-aminopyridin-4-ylmethylcarbonyloxy)prop 1-ynyl]-5-methoxycarbonyl methylphenyl}prop-2-ynyloxycarbonylamino)pyrrolidine-2-carboxylate, and the salts of these compounds.

A special embodiment of the invention are compounds of the formula 1, in which R1 is methoxy-carbonyl.

Another special embodiment of the invention are compounds of the formula 1, in which R2 is methoxycarbonyl.

Still another special embodiment of the invention are compounds of the formula 1, in which X1 and X2 are identical and are amino.

A further special embodiment of the invention are compounds of the formula 1, in which M is the following central building block 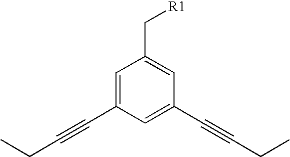
and R1 is methoxycarbonyl.

Another further special embodiment of the invention are compounds of the formula 1, in which M is the following central building block 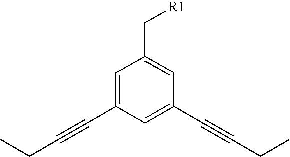
R1
R1 is methoxycarbonyl,
B1 and B2 are identical and are —O— and
X1 and X2 are identical and are amino.

Still a further special embodiment of the invention are compounds of the formula 1, in which M is the following central building block 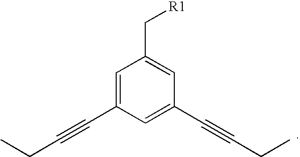
R1 is methoxycarbonyl,
B1 and B2 are identical and are —O—,
X1 and X2 are identical and are amino,
   and R2 is methoxycarbonyl.

The compounds of the formula 1 are composed of a large number of building blocks (M, B1, B2, B3, B4, B5, B6, K1, K2, X1, X2, Z1 and Z2). They can be synthesized in principle starting from any of these building blocks. Compounds of the formula 1 with a substantially symmetrical structure are appropriately assembled starting from the central building block M, while synthesis of predominantly unsymmetrical compounds of the formula 1 may advantageously start from one of the end groups K1 or K2.

The linkage of the building blocks always takes place according to the same pattern known to the skilled worker.

The skilled worker is aware that the compounds of the formula 1 either can be assembled building block by building block, or that initially larger fragments consisting of a plurality of individual building blocks can be produced and then assembled to give the complete molecule.

On the basis of the meanings which the individual building blocks of the compounds of the formula 1 may assume, ether [—O—], amide [—C(O)—NH—], carbamate [—O—C(O)—NH—] or carbamide bridges [—NH—C(O)—NH—] occur in the compounds of the formula 1.

The way in which such bridges are produced is known per se to the skilled worker, and suitable methods and starting compounds for preparing them are described for example in March, Advanced Organic Chemistry, Reactions, Mechanisms and Structure, Third Edition, 1985, John Wiley & Sons.

Ether bridges can be produced for example by the Williamson method.

There is also a large number of methods known for producing amide bridges. An example which may be mentioned here is reaction of acid chlorides with primary or secondary amines. Reference may also be made to all the methods developed for peptide chemistry.

Carbamate bridges can be produced for example by reacting chlorocarbonic esters with amines. The chlorocarbonic esters in turn can be assembled from alcohols and phosgene. A further variant for assembling carbamate bridges is the addition of alcohols onto isocyanates. Carbamide bridges can be prepared, for example, by reacting isocyanates with amines.

The synthesis of exemplary components of the formula 1 is depicted in reaction schemes 1-4 below.

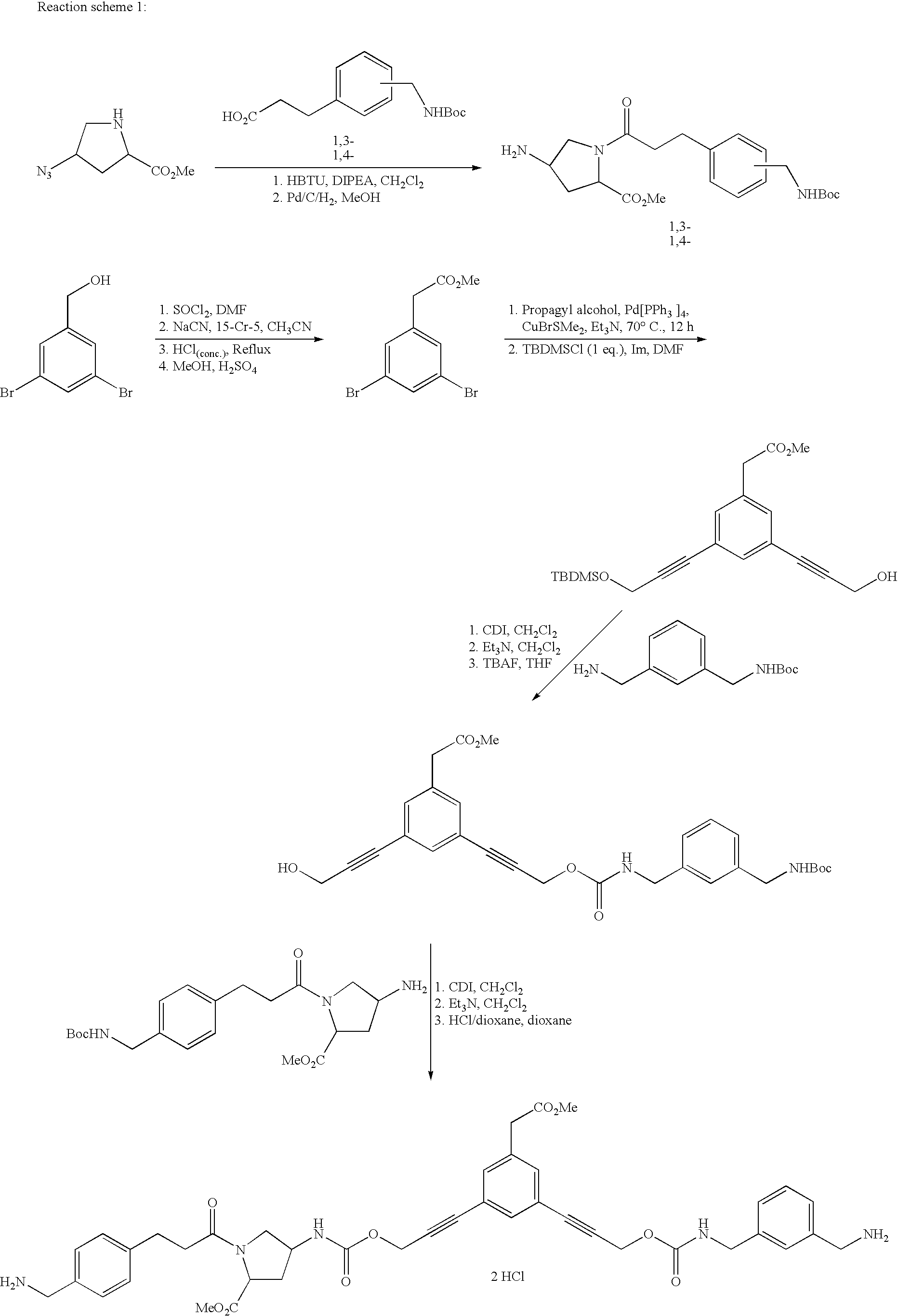

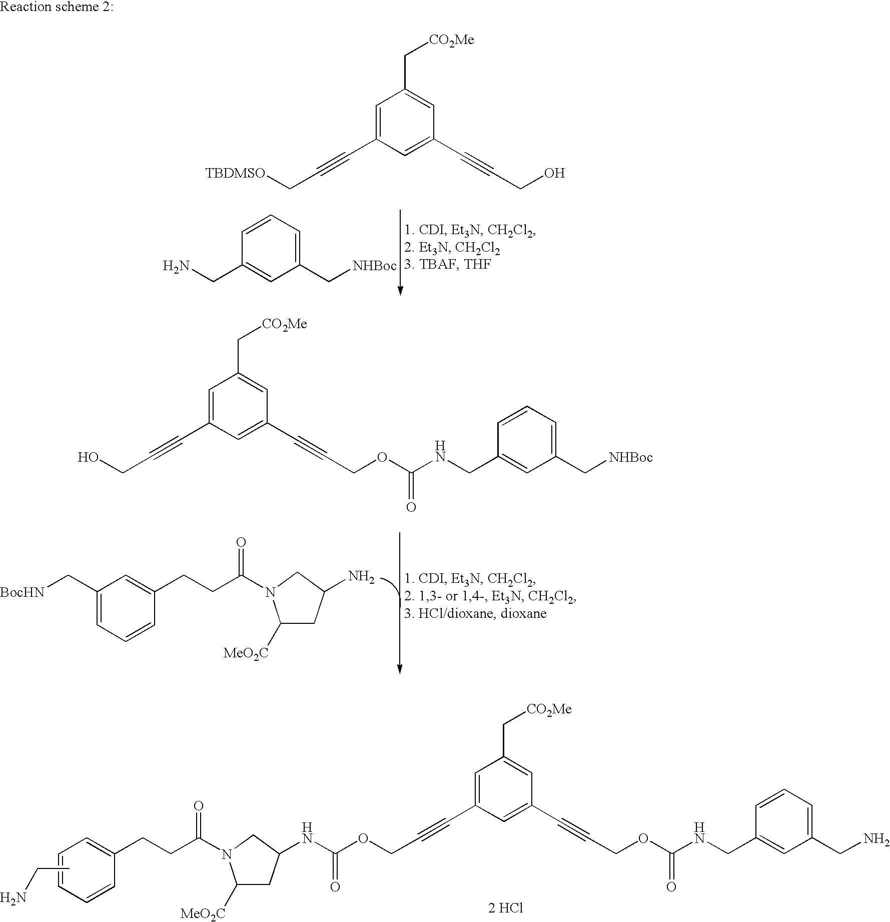

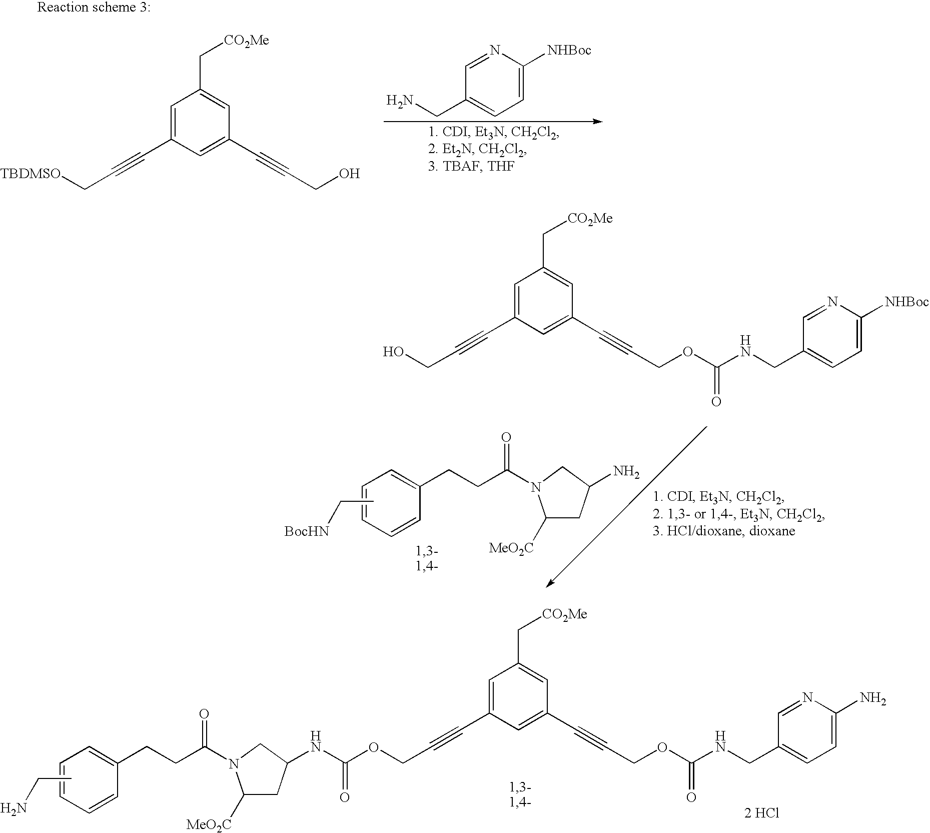

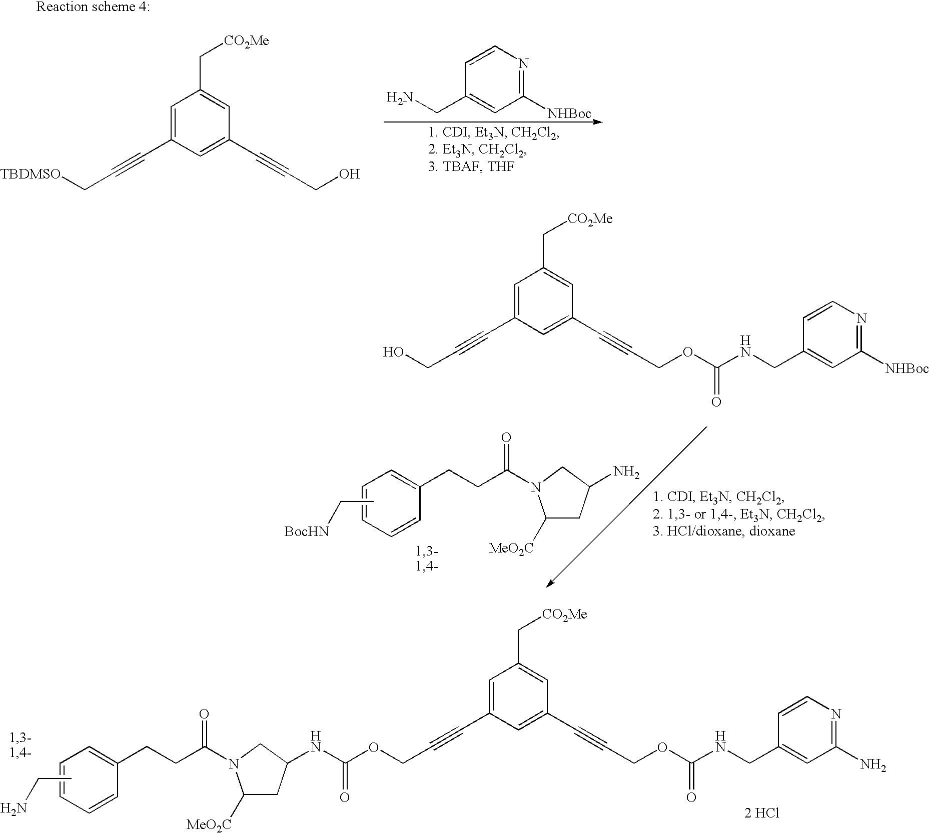

Further compounds of the formula 1 whose preparation is not explicitly described in the reaction schemes can be prepared in an analogous way or in a way familiar per se to the skilled worker using conventional process techniques.

A starting compound which can be used for compounds of the formula 1 para-linked on the central building block M is, for example, methyl (2,5-dibromophenyl)acetate.

The skilled worker is additionally aware that it may be necessary, in the case of a plurality of reactive centers on a starting compound or intermediate, to block one or more reactive centers temporarily by protective groups in order to allow a reaction to proceed specifically at the desired reaction center. A detailed description of the use of a large number of proven protective groups is to be found, for example, in T. W. Greene, Protective Groups in Organic Synthesis, John Wiley & Sons, 1991.

The isolation and purification of the substances of the invention takes place in a manner known per se, for example by the solvent being distilled off in vacuo, and the resulting residue being recrystallized from a suitable solvent or subjected, to one of the conventional purification methods such as, for example, column chromatography on suitable support material.

Salts are obtained by dissolving the free compound in a suitable solvent (e.g. a ketone such as acetone, methyl ethyl ketone or methyl isobutyl ketone, an ether such as diethyl ether, tetrahydrofuran or dioxane, a chlorinated hydrocarbon such as methylene chloride or chloroform, or a low molecular weight aliphatic alcohol such as ethanol or isopropanol) which contains the desired acid or base, or to which the desired acid or base is subsequently added. The salts are obtained by filtration, reprecipitation, precipitation with a nonsolvent for the addition salt or by evaporating the solvent. Resulting salts can be converted by basification or by acidification into the free compounds which can in turn be converted into the salts. It is possible in this way to convert pharmacologically unacceptable salts into pharmacologically acceptable salts.

In the following examples, the abbreviation RT stands for room temperature, h for hours, min for minutes and calc. for calculated.

The compounds mentioned by way of example and their salts are a preferred aspect of the invention.

### EXAMPLES

Final Compounds:

General Method

A solution of the particular Boc-protected bivalent compound (A1-A6; 1.0 mmol) in dioxane (12.5 ml) is mixed with a saturated solution of HCl in dioxane (12.5 ml) and stirred at RT for 8-10 h. The reaction mixture is then diluted with diethyl ether (10 ml) and the resulting precipitate is filtered off and washed with diethyl ether (3×5 ml). Drying in vacuo results in the title compounds (final compounds 1-6) as colorless solids.

### 1. Methyl 4-(3-{3-[3-(3-aminomethylbenzylcarbonyloxy)prop-1-ynyl]-5-methoxy-carbonyl-methylphenyl}prop-2-ynyloxycarbonylamino)-1-[3-(3-aminomethylphenyl)-propanoyl]-pyrrolidine-2-carboxylate dihydrochloride

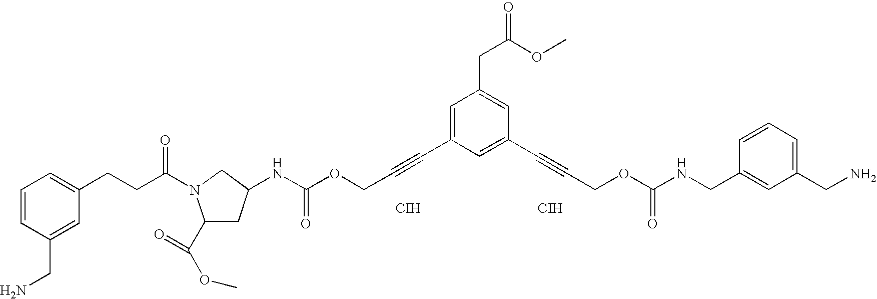

MS: calc.: C₄₁H₄₅N₅O₉ (751.8), found: [MH⁺] 752.3

### 2. Methyl 4-(3-{3-[3-(3-aminomethylbenzylcarbonyloxy)prop-1-ynyl]-5-methoxy-carbonyl-methylphenyl}prop-2-ynyloxycarbonylamino)-1-[3-(4-aminomethylphenyl)-propanoyl]-pyrrolidine-2-carboxylate dihydrochloride

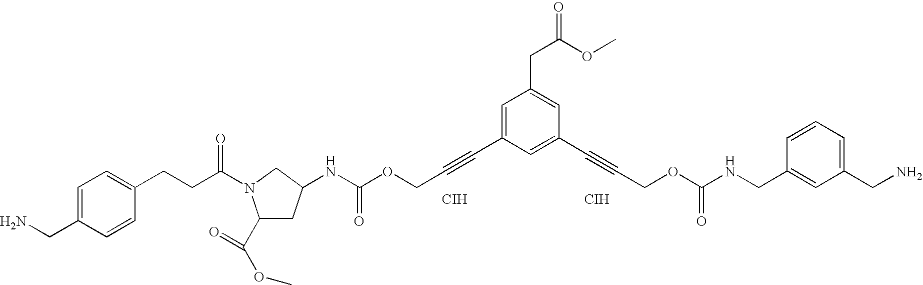

MS: calc: C₄₁H₄₅N₅₀₉ (751.8), found: [MH⁺]752.3

### 3. Methyl 1-[3-(3-aminomethylphenyl)propanoyl]-4-(3-{3-[3-(6-aminopyridin-3-ylmethyl-carbonyloxy)prop-1-ynyl]-5-methoxycarbonylmethylphenyl}prop-2-ynyloxycarbonyl-amino)pyrrolidine-2-carboxylate dihydrochloride

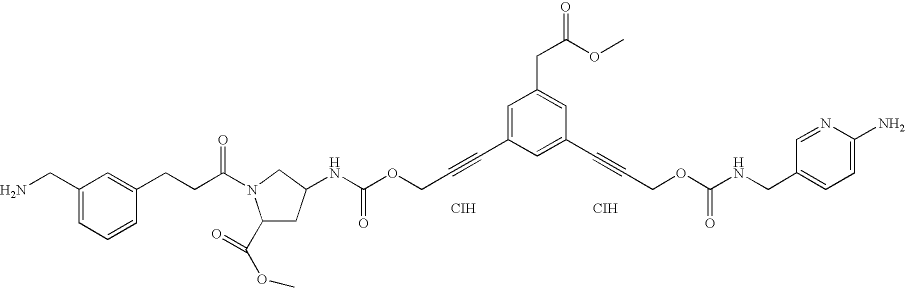

MS: calc.: C₃₉H₄₂N₆O₉ (738.8), found: [MH⁺] 739.3, [MNH₄⁺] 757.3

### 4. Methyl 1-[3-(4-amino methylphenyl)propanoyl]-4-(3-{3-[3-(6-aminopyridin-3-ylmethyl-carbonyloxy)prop-1-ynyl]-5-methoxycarbonylmethylphenyl}prop-2-ynyloxycarbonyl-amino)pyrrolidine-2-carboxylate dihydrochloride

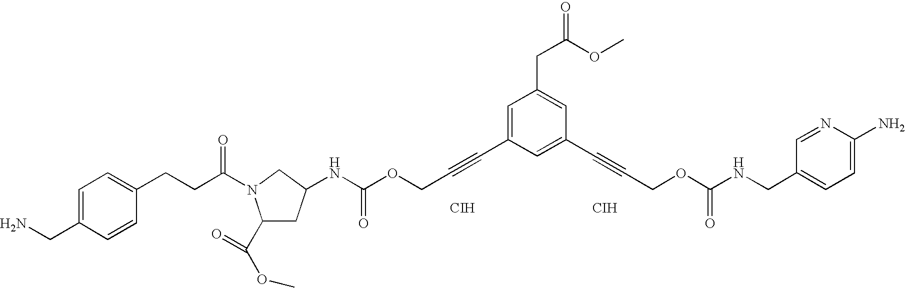

MS: calc.: C₃₉H₄₂N₆O₉ (738.8), found: [MH⁺] 739.3

### 5. Methyl 1-[3-(4-amino methylphenyl)propanoyl]-4-(3-{3-[3-(2-aminopyridin-4-ylmethyl-carbonyloxy)prop-1-ynyl]-5-methoxycarbonylmethylphenyl}prop-2-ynyloxycarbonyl-amino)pyrrolidine-2-carboxylate dihydrochloride

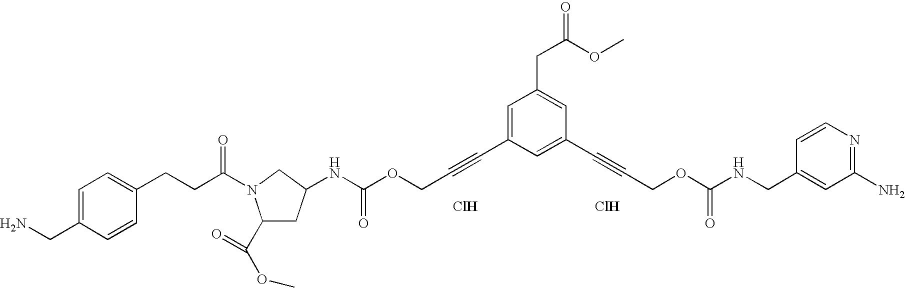

MS: calc: C₃₉H₄₂N₆O₉ (738.8), found: [MH⁺] 739.2

### 6. Methyl 1-[3-(3-aminomethylphenyl)propanoyl]-4-(3-{3-[3-(2-aminopyridin-4-ylmethyl-carbonyloxy)prop-1-ynyl]-5-methoxycarbonylmethylphenyl}prop-2-ynyloxycarbonyl-amino)pyrrolidine-2-carboxylate dihydrochloride

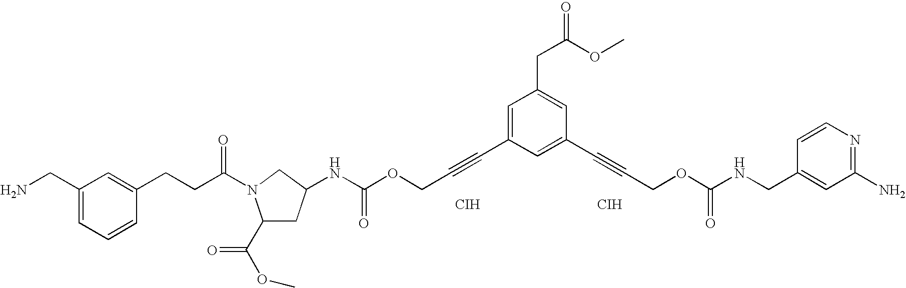 MS: calc: C₃₉H₄₂N₆₀₉ (738.8), found: [MH⁺]739.3
Starting Compounds and Intermediates:
General Method I:

N,N-Carbonyldiimidazole (0.19 g, 1.2 mmol) is added to a solution of the particular hydroxy compound A21-A23 in CH₂Cl₂ (10 ml). The reaction mixture is stirred at RT for 34 hand then diluted with CH₂Cl₂ (10 ml) and extracted with a half-saturated aqueous NaCl solution (15 ml). The organic phase is dried over MgSO₄, filtered and concentrated in vacuo. The resulting residue is dissolved in CH₂Cl₂ (10 ml), and the particular Boc-protected intermediate A7 or A8 is added. The reaction mixture is stirred at RT overnight, DMF (4 ml) is added, and stirring is continued at 55° C. for 8 h. The reaction solution is then diluted with CH₂Cl₂ (10 ml) and extracted with a half-saturated aqueous NH₄Cl solution (15 ml). The organic phase is dried over MgSO₄, filtered and concentrated in vacuo. Further purification takes place by flash chromatography [Tol/Ac (8:2)] and affords the title compounds (A1-A6).

### A1. Methyl 1-[3-(3-tert-butoxycarbonylaminomethylphenyl)propanoyl]-4-[3-(3-{3-[3-(tert-butoxycarbonylaminomethyl)benzylcarbamoyloxy]prop-1-ynyl}-5-methoxy-carbonyl-methylphenyl)prop-2-ynyloxycarbonylamino]pyrrolidine-2-carboxylate

Compound A21 (0.2 g, 0.38 mmol) is reacted by general method I with N,N-carbonyldiimidazole (95 mg, 0.58 mmol) and compound A7 (0.17 g, 0.42 mmol). The title compound (340 mg) is isolated as a colorless solid foam. TLC [Tol/Ac (7:3)], Rr 0.47.

MS: calc.: C₅₁H₆₁N₅O₁₃ (952.1), found: [MNH₄⁺] 968.9 and [MNa⁺] 974.3

### A2. Methyl 1-[3-(4-tert-butoxycarbonylaminomethylphenyl)propanoyl]-4-[3-(3-(3-[3-(tert-butoxycarbonylaminomethyl)benzylcarbamoyloxy]prop-1-ynyl)-5-methoxy-carbonyl-methylphenyl)prop-2-ynyloxycarbonylamino]pyrrolidine-2-carboxylate

Compound A21 (0.3 g, 0.58 mmol) is reacted by general method I with N,N-carbonyldiimidazole (0.14 g, 0.86 mmol) and compound A8 (0.28 g, 0.69 mmol). The title compound (177 mg) is obtained as a colorless solid foam. TLC [Tol/Ac (9:1)], R_{f}=0.44.

MS: calc.: C₅₁H₆₁N₅O₁₃ (952.1), found: [MNH₄⁺] 968.9 and [MNa⁺] 974.3

### A3. Methyl 4-(3-{3-[3-(6-tert-butyloxycarbonylaminopyridin-3-ylmethylcarbonyloxy)prop-1-ynyl]-5-methoxycarbonylmethylphenyl}prop-2-ynyloxycarbonylamino)-1-{3-[3-tert-butyloxycarbonylaminomethyl)phenyl]propanoyl}pyrrolidine-2-carboxylate

Compound A23 (0.25 g, 0.49 mmol) is reacted by general method I with N,N-carbonyldiimidazole (125 mg, 0.77 mmol) and compound A7 (0.22 g, 0.54 mmol). The title compound (375 mg) is obtained as a colorless solid foam. TLC [Tol/Ac (7:3], R_{f}−0.41.

MS: calc.: C₄₉H₅₈N₆O₁₃ (939.0), found: [MH⁺] 939.1 and [MNa⁺] 961.1

### A4. Methyl 4-(3-{3-[3-(6-tert-butyloxycarbonylaminopyridin-3-ylmethylcarbonyloxy)prop-1-ynyl]-5-methoxycarbonylmethylphenyl}prop-2-ynyloxycarbonylamino)-1-{3-[4-tert-butyloxycarbonylaminomethyl)phenyl]propanoyl}pyrrolidine-2-carboxylate

Compound A23 (0.29 g, 0.58 mmol) is reacted by general method I with N,N-carbonyldiimidazole (0.14 mg, 0.87 mmol) and compound A8 (0.26 g, 0.63 mmol). The title compound (206 mg) is obtained as a colorless solid foam. TLC [Tol/Ac (7:3], R_{f}=0.35.

MS: calc.: C₄₉H₅₈N₅O₁₃ (939.0), found: [MH⁺] 939.1 and [MNa⁺] 961.2

### A5. Methyl 4-(3-{3-[3-(2-tert-butyloxycarbonylaminopyridin-4-ylmethylcarbonyloxy)prop-1-ynyl]-5-methoxycarbonylmethylphenyl}prop-2-ynyloxycarbonylamino)-1-{3-[4-(tert-butyloxycarbonylaminomethyl)phenyl]propanoyl}pyrrolidine-2-carboxylate

Compound A22 (0.3 g, 0.59 mmol) is reacted by general method I with N,N-carbonyldiimidazole (0.15 g, 0.93 mmol) and compound A8 (0.24 g, 0.59 mmol). The title compound (160 mg) is isolated as a colorless solid foam. TLC [Tol/Ac (7:3)], R_{f}=0.39.

MS: calc: C₄₉H₅₈N₆O₁₃ (939.0), found: [MH⁺] 939.1 and [MNa⁺] 961.2

### A6. Methyl 4-(3-{3-[3-(2-tert-butyloxycarbonylaminopyridin-4-ylmethylcarbonyloxy)prop-1-ynyl]-5-methoxycarbonylmethylphenyl}prop-2-ynyloxycarbonylamino)-1-{3-[3-(tert-butyloxycarbonylaminomethyl)phenyl]propanoyl}pyrrolidine-2-carboxylate

Compound A22 (0.25 g, 0.49 mmol) is reacted by general method I with N,N-carbonyldiimidazole (125 mg, 0.75 mmol) and compound A7 (0.20 g, 0.49 mmol). The title compound (173 mg) is obtained as a colorless solid foam. TLC [Tol/Ac (7:3)], R_{f}=0.38.

MS: calc.: C₄₉H₅₈N₆O₁₃ (939.0), found: [MH⁺] 939.1 and [MNa⁺] 961.2

### A7. Methyl 4-amino-1-{3-[3-(tert-butoxycarbonylaminomethyl)phenyl]propanoyl}pyrrolidine-2-carboxylate

4.7 g (10.9 mmol) of 4-azido-1-[3-(3-tert-butyloxycarbonylaminomethylphenyl)propionyl]proline methyl ester (A24) are dissolved in 70 ml of methanol and, after addition of 0.5 g of Pd/C (10%), hydrogenated. After the reaction Is complete, the catalyst is filtered off with suction and the filtrate Is concentrated in vacuo. Drying under high vacuum results in 3.8 g of the title compound as a colorless solidified foam. The mass spectrum shows the molecular peak MH⁺ at 406 Da.

### A8. Methyl 4-amino-1-{3-[4-(tert-butoxycarbonylaminomethyl)phenyl]propanoyl}pyrrolidine-2-carboxylate

6.27 g (14.5 mmol) of 4-azido-1-[3-(4-tert-butyloxycarbonylaminomethylphenyl)propionyl]proline methyl ester (starting compound A28) are dissolved in 200 ml of methanol and, after addition of 0.6 g of Pd/C (10%), hydrogenated. After the reaction is complete, the catalyst is filtered off with suction and the filtrate is concentrated in vacuo. Drying under high vacuum results in 5.47 g of the title compound as a colorless solidified foam. The mass spectrum shows the molecular peak MH⁺ at 406 Da.

### A9. 3,5-Dibromobenzyl chloride

3,5-Dibromobenzyl alcohol (9.8 g, 36.9 mmol) is dissolved in DMF (100 ml) and, while stirring, thionyl chloride (5 ml) is slowly added dropwise. After 30 m in, the reaction mixture is concentrated in vacuo and the residue is taken up in EA (150 ml). For work up, the organic phase is washed with ice-water (50 ml) and then with half-saturated aqueous NaCl solution (50 ml). The organic phase is dried over MgSO₄, filtered and concentrated in vacuo. The title compound (10.4 g) is obtained as a yellow solid. TLC [PE/EA (95:05)], R_{f}=0.69.

### A10. 3,5-Dibromobenzyl cyanide

A solution of 3,5-dibromobenzyl chloride (10.4 g, 36.4 mmol) in acetonitrile (40 ml) is added dropwise to a stirred suspension of NaCN (4.9 g, 99.8 mmol) and 15-crown-5 (5 ml) in acetonitrile (40 ml). The reaction mixture is stirred at RT overnight. The reaction mixture is taken up in EA (150 ml) and washed with half-saturated aqueous NaCl solution (2×70 ml). The organic phase is dried over MgSO₄, filtered and concentrated in vacuo. Subsequent purification by flash chromatography [PE/EA (9:1)] affords the title compound (7.25 g) as a yellow solid. TLC [PE/EA (9:1)], R_{f}=0.43

MS: calc.: C₈H₅Br₂N (274.9), found: [M⁺] 275.0

### A11. 3,5-Dibromobenzylic acid

3,5-Dibromobenzyl cyanide (18.6 g, 67.5 mmol) is suspended in concentrated HCl (350 ml) and stirred at RT overnight. The reaction mixture is then brought under reflux for 4 h. The solid is filtered off, washed with H₂O and dissolved in 2N NaOH (300 ml). The aqueous phase is extracted with EA (3×150 ml). The aqueous phase is then acidified with half-concentrated aqueous HCl. The resulting precipitate is filtered off, washed with water and dried. The title compound (14.2) is obtained as a colorless powder. TLC [PE:EA (8.5:1.5)], R_{f}=0.15.

MS: calc.: C₈H₆O₂Br₂ (293.9), found: [M⁺] 294

### A12. Methyl 3,5-dibromobenzylate

3,5-Dibromobenzylic acid (6.9 g, 23.6 mmol) is dissolved in MeOH (150 ml) and stirred at 0° C. While stirring, conc. H₂SO₄ (21.5 ml) is added dropwise, and the mixture is then heated under reflux for 3 h. After concentration in vacuo, the resulting residue is dissolved in CH₂Cl₂ (150 ml) and extracted with half-concentrated aqueous NaCl solution (2×50 ml). The organic phase is dried over MgSO₄, filtered and concentrated in vacuo. The title compound (7.19 g) is obtained as colorless crystals. TLC [PE/EA (8.5:1.5)], R_{f}=0.71.

MS: calc.: C₈H₈O₂Br₂ (308.0), found: [M+] 308.0

### A13. Methyl[3,5-bis(3-hydroxyprop-1-ynyl)phenyl]acetate

Methyl 3,5-dibromobenzylate (4.0 g, 12.9 mmol) is dissolved in Et₃N (90 ml), and CuBr.SMe₂ (0.29 g) is added, and the mixture is stirred at RT for 10 min. Then Pd(PPh₃)₄ (0.69 g) is added and the mixture is stirred at RT for a further 10 min. Propagyl alcohol (3.8 ml, 64.4 mmol) is added dropwise to the reaction mixture, which is stirred at RT for 30 min and then at 80° C. for 4 h. The aqueous mixture is then mixed with half-saturated aqueous NH₄Cl solution (200 ml) and extracted with CH₂Cl₂ (3×100 ml). The organic phase is dried over MgSO₄, filtered and concentrated in vacuo. Further purification by flash chromatography [Tol/Ac (8:2)] yields the title compound (3.11 g) as a pale yellow oil. TLC [Tol/Ac (75:25)], R_{f}=0.43.

MS: calc.: C₁₄H₁₄O₄ (258.3), found: [MNH₄⁺] 276.0

### A14. Metyl{3-[3-(tert-butyldimethylsilanyloxy)prop-1-ynyl]-5-(3-hydroxyprop-1-ynyl)phenyl}-acetate

Methyl[3,5-bis(3-hydroxyprop-1-ynyl)phenyl]acetate (8.9 g, 34.5 mmol) is dissolved in DMF (250 ml) and, at 0° C., imidazole (3.6 g, 53.2 mmol) is added and subsequently a solution of TBDMSCI (3.6 g, 24.2 mmol) in DMF (100 ml) is slowly added dropwise. After about 1 h, the reaction solution is allowed to warm to RT, aqueous NH₄Cl solution is added, and the mixture is extracted with EA (3×100 ml). The combined organic phases are dried over MgSO₄, filtered and concentrated in vacuo. Subsequent purification by flash chromatography [Tol/Ac (9:1) affords the title compound (4.8 g) as yellow liquid. TLC [Tol/Ac (9:1)], R_{f}=0.50.

MS: calc.: C₂, H₂₈O₄Si (372.54), found: [MNH₄⁺] 390.1

### A 15. 3-N-tert-Butoxycarbonylaminomethylbenzylamine

The title compound was prepared by the method of Cross, R.; Duener, G.; Goebel, M.; Michael, W. Liebigs Ann. Chem. 1994, 1, 49-58.

### A 16. 4-Aminomethyl-2-N-tert-butoxycarbonylaminopyridine

The title compound is prepared in accordance with the methods in the international patent application WO00/14097.

### A 17. 5-Aminomethyl-2-N-tert-butoxycarbonylaminopyridine

The title compound is prepared in accordance with the methods in the international patent application WO00/14097.

General method II:

N,N-Carbonyldiimidazole (0.25 g, 1.5 mmol) is added to a solution of compound A14 (1.0 mmol) in CH₂Cl₂ (8 ml) and stirred at RT for 2-3 h. The reaction solution is diluted with CH₂Cl₂ (8 ml) and extracted with half-saturated aqueous NaCl solution (12 ml). The organic phase is dried over MgSO₄, filtered and concentrated in vacuo. The resulting residue is dissolved in absolute CH₂Cl₂ (8 ml) and, after addition in each case of the appropriate head group building block (A15-A17; 1.1 mmol), stirred at RT overnight. The reaction solution is then diluted with CH₂Cl₂ (8 ml) and extracted with half-saturated aqueous NH₄Cl solution (12 ml). The organic phase is dried over MgSO₄, filtered and concentrated in vacuo. Further purification takes place by flash chromatography [Tol/Ac (9:1)] and affords the title compounds A18-A20.

### A18. Methyl[3-{3-[3-(tert-butoxycarbonylaminomethyl)benzylcarbonyloxy]-prop-1-ynyl}-5-(3-tert-butyldimethylsilanyloxyprop-1-ynyl)phenyl]acetate

Compound A14 (2.8 g, 7.6 mmol) is reacted by general method II with N,N-carbonyldiimidazole (2.15 g, 13.3 mmol) and compound A15 (2.75 g, 11.6 mmol). The title compound (4.4 g) is obtained as a colorless oil. TLC [Tol/Ac (9:1), R_{f}=0.61.

MS: calc.: C₃₅H₄₆N₂O₇Si (634.4), found: [MH⁺] 634.5, [MNH₄⁺] 651.9 and [MNa⁺] 657.2

### A19. Methyl[3-[3-(2-tert-butoxycarbonylaminopyridin-4-yl-methylcarbonyloxy)prop-1-ynyl]-5-(3-tert-butyldimethylsilanyloxyprop-1-ynyl)phenyl]acetate

Compound A14 (1.0 g, 2.7 mmol) is reacted by general method II with N,N-carbonyldiimidazole (0.66 g, 4.1 mmol) and compound A16 (0.6 g, 2.7 mmol). The title compound (1.7 g) is obtained as a colorless oil. TLC [Tol/Ac (9:1), R_{f}=0.33.

MS: calc.: C₃₃H₄₃N₃O₇Si (621.8), found: [MH⁺] 621.9

### A20. Methyl[3-[3-(6-tert-butoxycarbonylaminopyridin-3-ylmethylcarbonyloxy)prop-1-ynyl]-5-(3-tert-butyldimethylsilanyloxyprop-1-ynyl)phenyl]acetate

Compound A14 (1.2 g, 3.2 mmol) is reacted by general method II with N,N-carbonyldiimidazole (0.8 g, 4.9 mmol) and compound A17 (0.8 g, 3.5 mmol). The title compound (1.79 g) results as a colorless oil. TLC [Tol/Ac (9:1)], R_{f}=0.47.

MS: calc.: C₃₃H₄₃N₃O₇Si (621.8), found: [MH⁺] 622.0 und [MNa⁺] 644.0

General Method III:

A 1M solution of tetrabutylammonium fluoride in THF (1.1 ml, 1.1 mmol) is added to a solution of the respective compounds A18-A20 (1.0 ml) in THF (15 ml) and stirred at RT for 1-1.5 h. The reaction solution is then diluted with CH₂Cl₂ (30 ml) and extracted with a half-saturated aqueous NH₄Cl solution (30 ml). The organic phase is dried over MgSO₄, filtered and concentrated in vacuo. Further purifycation takes place by flash chromatography [Tol/Ac (8:2)]. The title compounds A21-A23 are obtained as colorless and slightly yellowish solids.

### A21. Methyl[3-{3-[3-(tert-butoxycarbonylaminomethyl)benzylcarbonyloxy]prop-1-ynyl}-5-(3-hydroxyprop-1-ynyl)phenyl]acetate

Compound A18 (4.35 g, 6.85 mmol) is reacted by general method III with a 1M solution of tetrabutylammonium fluoride in THF (7.8 ml, 7.8 mmol). The title compound (2.0 g) is obtained as a pale yellowish solid. TLC [Tol:Ac (9:1)], R_{f}=0.15.

MS: calc.: C₂₉H₃₂N₂O₇ (520.7), found: [MNH₄⁺] 537.9 and [MNa⁺] 543.2

### A22. Methyl[3-[3-(2-tert-butoxycarbonylaminopyridin-4-ylmethylcarbonyloxy)prop-1-ynyl]-5-(3-hydroxyprop-1-ynyl)phenyl]acetate

Compound A19 (2.07 g, 3.33 mmol) is reacted by general method III with a 1M solution of tetrabutylammonium fluoride in THF (3.7 ml, 3.7 mmol). The title compound (0.95 g) is obtained as a pale yellowish solid. TLC [Tol/Ac (8:2)], R_{f}=0.28.

MS: calc.: C₂₇H₂₉N₃O₇ (507.6), found: [MH⁺] 507.9 and [MNa⁺] 529.9

### A23. Methyl[3-[3-(6-tert-butoxycarbonylaminopyridin-3-yl-methylcarbonyloxy)prop-1-ynyl]-5-(3-hydroxyprop-1-ynyl)phenyl]acetate

Compound A20 (1.75 g, 2.81 mmol) is reacted by general method III with a 1M solution of tetrabutylammonium fluoride in THF (3.1 ml, 3.1 mmol). The title compound (0.56 g) is obtained as a colorless solid. TLC [Tol:Ac (8:2)], R_{f}=0.32.

MS: calc.: C₂₇H₂₉N₃O₇ (507.6), found: [MH⁺] 507.9 and [MNa⁺] 530.0

### A24. 4-Azido-1-[3-(3-tert-butyloxycarbonylaminomethyl)phenyl)propanoyl]pyrrolidine-2-carboxylate

1.61 g (5.7 mol) of 3-[3-(tert-butyloxycarbonylaminomethyl)phenyl]propionic acid (starting compound A25) are dissolved in 14 ml of CH₂Cl₂, and 2.1 ml of DIPEA are added. After stirring for 5 min., 2.2 g (5.7 mmol) of HBTU are added and, after a further 5 min., 1.0 g (4.8 mmol) of (2S,4S)₄-azidoproline methyl ester hydrochloride. After stirring at RT overnight, ethyl acetate and water are added and the phases are separated. The organic phase is washed once each with 1N sodium hydroxide solution, 1N hydrochloric acid solution, saturated sodium bicarbonate solution and saturated brine. Drying over magnesium sulfate is followed by concentration and drying under high vacuum. Further purification takes place by chromatography [Tol/Ac (8:2)] on a silica gel column. The title compound (1.5 g) Is obtained as a colorless oil. TLC, silica gel (glass plates), [toluene/acetone (8:2)], R_{f}=0.31. The mass spectrum shows the molecular peak MNH₄⁺ at 449 Da.

### A25. 3-[3-(tert-Butyloxycarbonylaminomethyl]phenylpropionic acid

19.46 g of methyl 3-[3-(aminomethyl)phenyl]propionate hydrochloride (starting compound A26) are dissolved in 200 ml of dichloromethane and, while stirring at 0° C., 27 ml of triethylamine and a solution of 16.8 g of di-tert-butyl dicarbonate in 10 ml of dichloromethane are successively added. After stirring at 0° C. for 1 h and at RT for a further 3 h, the reaction solution is washed twice with 0.1N hydrochloric acid solution and then with sodium bicarbonate solution and water, and dried over magnesium sulfate. Filtration is followed by concentration in vacuo, and the residue (13.5 g) is dissolved in 188 ml of tetrahydrofuran, and 38 ml of 2N sodium hydroxide solution are added. After stirring at RT overnight, 4N hydrochloric acid solution is used to neutralize, and the organic solvent is removed by distillation in vacuo. The resulting colorless precipitate is filtered off with suction, washed with water and dried under high vacuum. 12.8 g of the title compound are obtained, and its mass spectrum shows the molecular peak MNH₄⁺ at 297 Da.

### A26. Methyl 3-[3-(aminomethyl)phenyl]propionate hydrochloride

12.5 g of methyl (E)-3-(3-cyanophenyl)acrylate (starting compound A27) are dissolved in a mixture of 130 ml of methanol and 8 ml of acetic acid and hydrogenated over 1.3 g of palladium/carbon (10%) for 4 h. The catalyst is filtered off and the filtrate is concentrated in vacuo. The residue is stirred with ether and then a solution of hydrogen chloride in ether is added. The resulting precipitate is filtered off with suction, washed with ether and dried in vacuo. 19.5 g of the title compound are obtained. The mass spectrum shows the molecular peak MH⁺ at 194 Da.

### A27. Methyl (E)-3-(3-cyanophenyl)acrylate

7.31 ml (74.5 mmol) of methyl acrylate, 13.6 g (74.5 mmol) of 3-bromobenzonitrile and 6.6 g (74.5 mmol) of sodium acetate are suspended in 100 ml of DMF and heated at 120° C. for 30 min until a clear solution has formed. Then a solution of 4.0 g of palladium acetate and 21.0 g of tri-p-tolylphosphine in 5 ml of DMF is added dropwise to the reaction solution, and the mixture is stirred at 120° C. for 2 h. The reaction solution is then diluted with 500 ml of water, and the resulting precipitate is filtered off with suction. Drying under high vacuum and recrystallization from ethyl acetate/petroleum ether result in 12.6 g of the title compound. The mass spectrum shows the molecular peak M⁺/MH⁺ at 187 Da.

### A28. Methyl 4-azido-{1-[3-(4-tert-butyloxycarbonylaminomethyl)phenyl]-propanoyl}pyrrolidine-2-carboxylate

2.70 g (9.5 mmol) of 3-[4-(tert-butyloxycarbonylaminomethyl)phenyl]propionic acid (starting compound A29) are dissolved in 40 ml of DMF, and 2.7 ml of triethylamine are added. After stirring for 5 min, 3.63 g of HBTU are added and, after a further 5 min, 2 g of (2S,4S)₄-azidoproline methyl ester hydrochloride. After stirring at RT overnight, ethyl acetate and water are added, and the phases are separated. The organic phase is washed once each with 1N sodium hydroxide solution, 1N hydrochloric acid solution, saturated sodium carbonate solution and saturated brine. Drying over magnesium sulfate is followed by concentration and drying under high vacuum. 4.1 g of the title compound are obtained as a pale orange oil. The mass spectrum shows the molecular peak MNH₄⁺ at 449 Da.

### A29. 3-[4-(tert-Butyloxycarbonylaminomethyl)phenyl]propionic acid

4.65 g of methyl 3-[4-(aminomethyl)phenyl]propionate hydrochloride (starting compound A30) are dissolved in 20 ml of dichloromethane and, while stirring at 0° C., 6.17 ml of triethylamine and a solution of 4.62 g of di-tert-butyl dicarbonate in 10 ml of dichloromethane are successively added. After stirring at 0° C. for 1 h and at RT for a further 3 h, the reaction solution is washed twice with 0.1N hydrochloric acid solution and then with sodium bicarbonate solution and water, and dried over magnesium sulfate. Filtration is followed by concentration in vacuo, and the residue (5.6 g) dissolved in 50 ml of tetrahydrofuran, and 13.4 ml of 2N sodium hydroxide solution are added. After stirring at RT overnight, 6.7 ml of 4N hydrochloric acid solution is used to neutralize, and the organic solvent is removed by distillation in vacuo. The resulting colorless precipitate is filtered off with suction, washed with water and dried under high vacuum. 4.65 g of the title compound are obtained, and its mass spectrum shows the molecular peak MNH₄⁺ at 297 Da.

### A30. Methyl 3-[4-(aminomethyl)phenyl]propionate hydrochloride

5.6 g of methyl 3-[4-(hydroxyiminomethyl)phenyl]acrylate (starting compound A31) are dissolved in a mixture of 170 ml of methanol and 50 ml of acetic acid and hydrogenated over 0.5 g of palladium/carbon (10%) for 4 h. The catalyst is filtered off and the filtrate is concentrated in vacuo. The residue is stirred with ether and then a solution of hydrogen chloride in ether is added. The resulting precipitate is filtered off with suction, washed with ether and dried in vacuo. 4.65 g of the title compound are obtained. The mass spectrum shows the molecular peak MH⁺ at 194 Da.

### A31. Methyl 3-[4-(hydroxyiminomethyl)phenyl]acrylate

4.0 g of methyl 3-(4-formylphenyl)acrylate are dissolved in 40 ml of methanol and then 1.6 g of hydroxylamine hydrochloride and 1.9 g of sodium acetate are successively added. The mixture is stirred overnight and then diluted with 300 ml of water, and the resulting precipitate is filtered off with suction. Drying under high vacuum and recrystallization from ethyl acetate/petroleum ether result in 356 g of the title compound. The mass spectrum shows the molecular peak MH⁺ at 206 Da.

### INDUSTRIAL APPLICATION

The compounds of the invention have, as tryptase inhibitors, valuable pharmacological properties which make them immensely utilizable. Human tryptase is a serine protease which Is the predominant protein present in human mast cells. Tryptase comprises eight closely related enzymes (α1, α2, β1a, β1b, β2, β3, mMCP-7-like-1, mMCP-7-like-2; 85 to 99% sequence identity) (cf. Miller et al., J. Clin. Invest. 84 (1989) 1188-1195; Miller et al., J. Clin. Invest. 86 (1990) 864-870; Vanderslice et al., Proc. Natl. Acad. Sci., USA 87 (1990) 3811-3815; Pallaoro et al., J. Biol. Chem. 274 (1999) 3355-3362). However, only β-tryptases (Schwartz et al., J. Clin. Invest. 96 (1995) 2702-2710; Sakai et al., J. Clin. Invest. 97 (1996) 988-995) undergo intracellular activation and are stored in catalytically active form in secretory granules. Tryptase has some special properties by comparison with other known serine proteases such as, for example, trypsin or chymotrypsin (Schwartz et al., Methods Enzymol. 244, (1994), 88-100; G. H. Caughey, “Mast cell proteases in immunology and biology”, Marcel Dekker, Inc., New York, 1995). Tryptase from human tissue has a non-covalently linked tetrameric structure which must be stabilized by heparin or other proteoglycans in order to have proteolytic activity. Tryptase is released together with other inflammatory mediators such as, for example, histamine and proteoglycans when human mast cells are activated. It is therefore assumed that tryptase is involved in a number of disorders, in particular in allergic and inflammatory disorders, on the one hand because of the significance of mast cells in such disorders, and on the other hand because an increased tryptase content has been found in a number of such disorders. Thus, tryptase is thought to be associated inter alia with the following disorders: acute and chronic (especially inflammatory and allergen-induced) airway disorders of various etiologies (e.g. bronchitis, allergic bronchitis, bronchial asthma, COPD); interstitial pulmonary disorders; disorders based on allergic reactions with the upper airways (pharynx, nose) and the adjacent regions (e.g. paranasal sinuses, conjunctivae), such as, for example, allergic conjunctivitis and allergic rhinitis; arthritic diseases (e.g. rheumatoid arthritis); autoimmune diseases such as multiple sclerosis; also neurogenic inflammations, arteriosclerosis and cancer; additionally periodontitis, anaphylaxis, interstitial cystitis, dermatitis, psoriasis, scleroderma/systemic sclerosis, inflammatory bowel disorders (Crohn's disease, ulcerative colitis) and others. Tryptase appears in particular to be directly associated with the pathogenesis of asthma (Caughey, Am. J. Respir. Cell Mol. Biol. 16 (1997), 621-628; R. Tanaka, “The role of tryptase in allergic inflammation” in: Protease Inhibitors, IBC Library Series, 1979, sections 3.3.1-3.3.23).

The invention further relates to the compounds of the invention for use in the treatment and/or prophylaxis of disorders, especially of the disorders mentioned.

The invention likewise relates to the use of the compounds of the invention for producing pharmaceutical compositions employed for the treatment and/or prophylaxis of the disorders mentioned.

The invention further relates to pharmaceutical compositions which comprise one or more of the compounds of the invention for the treatment and/or prophylaxis of the disorders mentioned.

The pharmaceutical corn positions are produced by processes known per se and familiar to the skilled worker. The compounds of the invention (=active ingredients) are employed as pharmaceutical compositions either as such or, preferably, in combination with suitable pharmaceutical excipients, e.g. in the form of tablets, coated tablets, capsules, suppositories, plasters, emulsions, suspensions, gels or solutions, with the content of active ingredient advantageously being between 0.1 and 95%.

The skilled worker is aware of the excipients which are suitable for the desired pharmaceutical formulations on the basis of his expert knowledge. Besides solvents, gel formers, ointment bases and other active ingredient carriers it is possible to use, for example, antioxidants, dispersants, emulsifiers, preservatives, solubilizers or permeation promoters.

For the treatment of disorders of the respiratory tract, the compounds of the invention are preferably also administered by inhalation, preferably in the form of an aerosol, with the aerosol particles of a solid, liquid or mixed composition having a diameter of from 0.5 to 10 μm, advantageously from 2 to 6 μm.

The aerosols can be generated for example by pressure-operated nozzle nebulizers or ultrasonic nebulizers, but advantageously by metered aerosols operated by propellant gas or propellant gas-free use of micronized active ingredients from inhalation capsules.

Depending on the inhalation system used, the dosage forms comprise besides the active ingredients also the necessary excipients such as, for example, propellant gases (e.g. Frigen in the case of metered aerosols), surface-active substances, emulsifiers, stabilizers, preservatives, aromatic substances, fillers (e.g. lactose in the case of powder inhalers) or, where appropriate, further active ingredients.

For inhalation purposes there are a large number of available appliances with which aerosols of optimal particle size can be generated and administered using an inhalation technique which is as appropriate for the patient as possible. Besides the use of attachments (spacers, expanders) and pear-shaped containers (e.g. Nebulator®, Volumatic®) and automatic actuators (Autohaler®) for metered aerosols, there are, especially for powder inhalers, a number of available technical solutions (e.g. Diskhaler®, Rotadisk®, Turbohaler® or the Inhaler described in European patent application EP 0 504 321) with which optimal administration of active ingredient can be achieved.

For the treatment of dermatoses, the compounds of the invention are used in particular in the form of pharmaceutical compositions which are suitable for topical application. The pharmaceutical compositions are produced by mixing the compounds of the invention (=active ingredients) preferably with suitable pharmaceutical excipients and further processing to suitable pharmaceutical formulations. Examples of suitable pharmaceutical formulations which may be mentioned are dusting powders, emulsions, suspensions, sprays, oils, ointments, fatty ointments, creams, pastes, gels or solutions.

The pharmaceutical compositions of the invention are produced by processes known per se. The dosage of the active ingredients on systemic therapy (oral or i.v.) is between 0.1 and 10 mg per kilogram and day.

Biological Investigations

The documented pathophysiological effects of mast cell tryptase are brought about directly by the enzymatic activity of the protease. Accordingly, they are reduced or blocked by inhibitors which inhibit the enzymatic activity of tryptase. A suitable measure of the affinity of a reversible inhibitor for the target protease is the equilibrium dissociation constant Kᵢ of the enzyme/inhibitor complex. This value of Kᵢ can be determined via the influence of the inhibitor on the tryptase-induced cleavage of a chromogenic peptide-p-nitroanilide substrate or of a fluorogenic peptide-aminomethylcoumarin substrate.

Methods

The dissociation constants for the tryptase/inhibitor complexes are determined under equilibrium conditions in accordance with the general proposals of Bieth (Bieth J G, Pathophysiological Interpretation of kinetic constants of protease inhibitors, Bull. Europ. Physiopath. Resp. 16:183-195,1980) and the methods of Sommerhoff et al. (Sommerhoff C P et al., A Kazal-type Inhibitor of human mast cell tryptase: Isolation from the medical leech Hirudo medicinalis, characterization, and sequence analysis, Biol. Chem. Hoppe-Seyler 375: 685-694, 1994).

Human tryptase is prepared pure from lung tissue or is prepared by recombination; the specific activity of the protease which has been determined by titration is normally more than 85% of the theoretical value. Constant amounts of the tryptase are incubated in the presence of heparin (0.1-50 μg/ml) to stabilize the protease with increasing amounts of the inhibitors. After equilibrium has been reached between the reactants, the remaining enzymic activity is determined after addition of the peptide-p-nitroanilide substrate tos-Gly-Pro-Arg-pNA, whose cleavage is followed at 405 nm for 3 min. Alternatively, the remaining enzymatic activity can also be determined using fluorogenic substrates. The apparent dissociation constants Kₗₐₚₚ (i.e. in the presence of substrate) are then found by nonlinear regression by fitting the enzyme rates to the general equation for reversible inhibitors (Morrison J F, Kinetics of the reversible inhibition of enzyme catalysed reactions by tight-binding inhibitors, Biochim. Biophys. Acta 185, 269-286, 1969):
Vₗ/V₀=1−{Eₜ+Iₜ+Kₗₐₚₚ−[(Eₜ+Iₜ+Kₗₐₚₚ)²−4EₜIₜ]^{1/2}}/2Eₜ

In this, Vₗ and V₀ are the rates respectively in the presence and absence of the inhibitor and Eₜ and Iₜ are the concentrations of tryptase and of the inhibitor.

The apparent dissociation constants found for the compounds of the invention are evident from the following table A, in which the numbers of the compounds correspond to the numbers of the compounds in the examples [pKₗₐₚₚ=−logKₗₐₚₚ(mol/l)].

TABLE A
Inhibition of human tryptase

| Compound | pKᵢₐₚₚ |
|---|---|
| 1 | 8.62 |
| 2 | 8.27 |
| 3 | 7.80 |
| 4 | 6.57 |
| 5 | 6.00 |
| 6 | 7.19 |

1. A compound of the formula 1 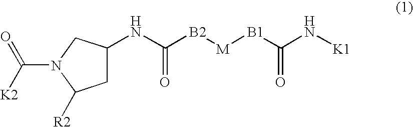
in which
M is a central building block selected from the following list 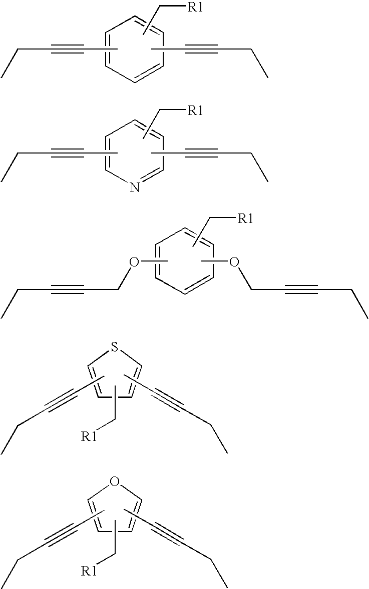
where
R1 is hydroxycarbonyl or 1-4C-alkoxycarbonyl,
B1 and B2 are identical or different and are —O—, —NH—, —O— (CH₂)ₘ—O— or —O—(CH₂)ₘ—NH—,
m is 1, 2, 3 or 4,
K1 is —B3-Z1—B5-X1,
K2 is —B4-Z2—B6-X2,
B3 is a bond or 1-2C-alkylene,
B4 is a bond or 1-2C-alkylene,
B5 is a bond or 1-2C-alkylene,
B6 is a bond or 1-2C-alkylene,
X1 and X2 are identical or different and are amino, aminocarbonyl, amidino or guanidino,
Z1 and Z2 are identical or different and are 5,2-pyridinylene, 3,6-pyridinylene, 4,2-pyridinylene, 1,3-phenylene, 1,4-phenylene, 1,3-cyclohexylene or 1,4-cyclohexylene,
R2 is —C(O)OR3 or —C(O)N(R4)R5 where
R3 is hydrogen, 1-4C-alkyl, 3-7C-cycloalkyl, 3-7C-cycloalkylmethyl or benzyl,
R4 and R5 are, independently of one another, hydrogen, 1-4C-alkyl, 3-7C-cycloalkyl or 3-7C-cycloalkylmethyl, or in which R4 and R5 together and with inclusion of the nitrogen atom to which they are bonded are a 1-pyrrolidinyl, 1-piperidinyl, 1-hexahydroazepinyl, 1-piperazinyl or 4-morpholinyl radical,
or a hydrate, solvate, salt, hydrate of a salt or solvate of a salt thereof.

2. A compound of the formula 1 as claimed in claim 1, in which
M is the following central building block 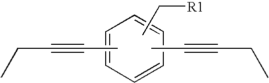
where
R1 is hydroxycarbonyl or 1-4C-alkoxycarbonyl,
B1 and B2 are identical or different and are —O— or —O— (CH₂)ₘ—O—,
m is 2,
K1 is —B3-Z1—B5-X1,
K2 is —B4-Z2—B6-X2,
B3 is a bond or 1-2C-alkylene,
B4 is a bond or 1-2C-alkylene,
B5 is a bond or 1-2C-alkylene,
B6 is a bond or 1-2C-alkylene,
X1 and X2 are identical or different and are amino or amidino,
Z1 is 3,6-pyridinylene, 4-2-pyridinylene, 1,3-phenylene or 1,4-phenylene,
Z2 is 1,3-phenylene or 1,4-phenylene,
R2 is —C(O)OR3 where
R3 is hydrogen, 1-4C-alkyl, 3-7C-cycloalkyl, 3-7C-cycloalkylmethyl or benzyl,
or a hydrate, solvate, salt, hydrate of a salt or solvate of a salt thereof.

3. A compound of the formula 1 as claimed in claim 1, in which
M is the central building block 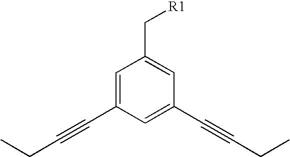
where
R1 is methoxycarbonyl,
B1 and B2 are identical and are —O—,
K1 is —B3-Z1—B5-X1,
K2 is —B4-Z2—B6-X2,
B3 is methylene,
B4 is ethylene,
B5 is a bond or methylene,
B6 is methylene,
X1 and X2 are identical and are amino,
Z1 is 3,6-pyridinylene, 4,2-pyridinylene, 1,3-phenylene or 1,4-phenylene,
Z2 1,3-phenylene or 1,4-phenylene,
R2 is methoxycarbonyl,
or a hydrate, solvate, salt, hydrate of a salt or solvate of a salt thereof.

4. A compound of the formula 1 as claimed 1, selected from the group consisting of
methyl 4-(3-{3-[3-(3-aminomethylbenzylcarbonyloxy)prop-1-ynyl]-5-methoxycarbonylmethylphenyl}-prop-2-ynyloxy-carbonylamino)-1-[3-(3-aminomethylphenyl)propanoyl]-pyrrolidine-2-carboxylate,
methyl 4-(3-{3-[3-(3-aminomethylbenzylcarbonyloxy)prop-1-ynyl]-5-methoxycarbonylmethylphenyl}-prop-2-ynyloxycarbonylamino)-1-[3-(4-aminomethylphenyl)propanoyl]-pyrrolidine-2-carboxylate,
methyl 1-[3-(3-aminomethylphenyl)propanoyl]-4-(3-{3-[3-(6-aminopyridin-3-ylmethylcarbonyloxy)prop-1-ynyl]-5-methoxycarbonylmethylphenyl}prop-2-ynyloxycarbonylamino)-pyrrolidine-2-carboxylate,
methyl 1-[3-(4-aminomethylphenyl)propanoyl]-4-(3-{3-[3-(6-aminopyridin-3-ylmethylcarbonyloxy)prop-1-ynyl]-5-methoxycarbonylymethylphenyl}prop-2-ynyloxycarbonylamino)-pyrrolidine-2-carboxylate,
methyl 1-[3-(4-aminomethylphenyl)propanoyl]-4-(3-{3-[3-(2-aminopyridine-4-ylmethylcarbonyloxy)prop-1-ynyl]-5-methoxycarbonylmethylphenyl}prop-2-ynyloxycarbonylamino)-pyrrolidine-2-carboxylate,
methyl 1-[3-(3-aminomethylphenyl)propanoyl]-4-(3-{3-[3-(2-aminopyridin-4-ylmethylcarbonyloxy)prop-1-ynyl]-5-methoxycarbonylmethylphenyl}prop-2-ynyloxycarbonylamino)-pyrrolidine-2-carboxylate,
and hydrates, solvates, salts, hydrates of the salts and solvates of the salts thereof.

5. A pharmaceutical composition comprising at least one compound of the formula 1 as claimed in claim 1, or a pharmaceutically acceptable hydrate, solvate, salt, hydrate of a salt or solvate of a salt thereof, together with a suitable pharmaceutical excipient.

6-9. (canceled)

10. A method for treating airway disorders in a patient in need thereof, comprising administering to said patient a therapeutically effective amount of a compound of formula 1 as claimed in claim 1, or a pharmaceutically acceptable hydrate, solvate, salt, hydrate of a salt or solvate of a salt thereof.

11. A method for treating dermatoses in a patient in need thereof, comprising administering to said patient a therapeutically effective amount of a compound of formula 1 as claimed in claim 1, or a pharmaceutically acceptable hydrate, solvate, salt, hydrate of a salt or solvate of a salt thereof.

12. A method for treating inflammatory bowel disorders in a patient in need thereof, comprising administering to said patient a therapeutically effective amount of a compound of formula 1 as claimed in claim 1, or a pharmaceutically acceptable hydrate, solvate, salt, hydrate of a salt or solvate of a salt thereof.
